(19) United States
(12) Patent Application Publication
(10) Pub. No.: US 2026/0224919 A1
(43) Pub. Date: Aug. 6, 2026

(54) ULTRASONIC STIMULATION SYSTEMS AND METHODS

(71) Applicant: The Alfred E. Mann Foundation for Scientific Research, Valencia, CA (US)

(72) Inventor: Brian V. Mech, Buffalo, MN (US)

(21) Appl. No.: 19/454,013

(22) Filed: Jan. 20, 2026

Related U.S. Application Data
(60) Provisional application No. 63/748,285, filed on Jan. 22, 2025.

(51) Int. Cl. A61N 7/02; A61B 8/00; A61B 8/08; A61N 1/05; A61N 1/36; A61N 7/00

(52) U.S. Cl. CPC A61N 7/02; A61B 8/085; A61B 8/4209; A61B 8/4472; A61B 8/5207; A61N 1/0502; A61N 1/36007; A61N 1/36017; A61N 1/36031; A61N 2007/0021; A61N 2007/0052; A61N 2007/0082

(57) ABSTRACT

The present disclosure generally relates to ultrasonic stimulation systems and methods of using the same. In particular, the present disclosure relates to external stimulation systems, comprising, e.g., one or more ultrasonic, electrical, and/or magnetic stimulators, which can be used to treat patients having an underactive bladder, and other conditions.

### CROSS-REFERENCE TO RELATED APPLICATION

The present application claims the benefit of priority to U.S. Provisional Application No. 63/748285, filed on Jan. 22, 2025, the entire contents of which is hereby incorporated by reference.

### TECHNICAL FIELD

The present disclosure generally relates to ultrasonic stimulation systems and methods of using the same. In particular, the present disclosure relates to external stimulation systems, comprising, e.g., one or more ultrasonic, electrical, or magnetic stimulators, which can be used to treat patients having an underactive bladder, and other conditions.

### BACKGROUND

Underactive bladder (“UAB”) or more formally detrusor underactivity, is a chronic, complex and often debilitating condition. UAB is characterized by a contraction of the detrusor muscle with reduced strength and/or duration resulting in prolonged bladder emptying and/or an inability to completely empty the bladder within a normal time span. Patients with UAB hold unusually large amounts of urine but have a diminished sense of when the bladder is full and are not able to contract the muscles sufficiently and as forcefully as they should, resulting in incomplete bladder emptying. UAB can be observed in many neurological conditions and myogenic failure. Management of UAB is focused on prevention of upper tract damage, avoidance of overdistension, and reduction of residual urine. Scheduled voiding, double voiding, α-adrenergic blockers, and intermittent self-catheterization are typical conservative treatment options. However, pharmacological intervention appears to provide little benefit, and is associated with undesirable side effects. See, e.g., Miyazato, et al., “The other bladder syndrome: underactive bladder.” Reviews in Urology 15.1 (2013):11. Sacral nerve stimulation via an implanted pulse generator (“IPG”) has been found to reduce symptoms of UAB in some patients. See Al-Zahrani, et al. “Long-term outcome and surgical interventions after sacral neuromodulation implant for lower urinary tract symptoms: 14-year experience at 1 center.” The Journal of urology 185.3 (2011): 981-986.

### SUMMARY OF SELECTED ASPECTS

Treatment options for UAB are currently limited. For example, pharmacological intervention has been shown to provide limited benefits and is associated with undesirable side effects. Surgical intervention (e.g., implantation of an IPG for neuromodulation) has been found to be successful in reducing UAB symptoms for some patients. However, implantation of a medical device is an invasive procedure and is non-ideal in view of the extended recovery time, cost, and potential for complications. Moreover, neither of these options have proven to provide consistent and effective reduction in UAB symptoms. Thus, there remains a need for alternative solutions for treating UAB. The present disclosure addresses this need by providing new systems and methods for treating UAB using ultrasonic, magnetic, electrical, and/or mechanical stimulation delivered from one or more external stimulators. These systems and methods provide various advantages and improvements over known systems (e.g., surgically implanted IPGs) and pharmaceutical intervention, by providing a cost-effective and non-invasive treatment option that can be customized for each patient in order to provide an effective personalized treatment regimen. In some aspects, the aforementioned features result in improved efficiency, reduced costs, and an improvement to the quality of life of patients suffering from UAB. Various other benefits and advantages shall become apparent in view of the present disclosure and the accompanying drawings.

In a first general aspect the disclosure provides a system for treating UAB, comprising one or more external stimulators, wherein each comprises a housing and a transducer, and is configured to deliver ultrasonic, electrical, or magnetic stimulation to a nerve of a subject; a controller, configured to communicate with the one or more external stimulators, and to set or modify one or more parameters of the delivery of the ultrasonic, electrical, and/or magnetic stimulation by the one or more external stimulators; wherein at least one of the one or more external stimulators is capable of delivering stimulation to a pudendal visceral, a parasympathetic, and/or a sympathetic nerve of a proximal urethra of a human subject when placed on or in proximity to a skin surface of the human subject.

In some aspects, the one or more external stimulators comprise at least one transducer configured to deliver ultrasonic stimulation.

In some aspects, the at least one transducer configured to deliver ultrasonic stimulation is configured to deliver stimulation a) at a frequency of about 150-750 kHz, b) with a spatial peak temporal average intensity below 100 W/cm², c) having a variable pulse repetition frequency, and/or d) having a mechanical index below 1.9. In some aspects, stimulation is provided using a duty cycle of 0-100% (e.g., 10, 20, 30, 40, 50, 60, 70, 80, 90, or 100%, or a percentage within a range bounded by any pair of the foregoing percentages).

In some aspects, at least one of the one or more external stimulators comprise a first transducer configured to deliver ultrasonic stimulation, and a second transducer configured to transmit and receive sound waves (e.g., for imaging purposes) at one or more frequencies within a range of 2 to 15 MHz (e.g., at 2, 3, 4, 5, 6, 7, 8, 9, 10, 11, 12, 13, 14, or 15 MHz, or at a frequency within a range bounded by any pair of the foregoing frequencies). In some aspects, a single transducer may be used both to deliver stimulation and for imaging.

In some aspects, the controller is configured to detect or identify one or more anatomical features of the human subject based on sound waves received by the second transducer (or the first transducer in the case where a single transducer is used for stimulation and imaging). The anatomical feature may comprise, e.g., the subject's urethra, bladder, and/or the junction of the urethra and the bladder, in the case of a male or female subject, and the prostate gland in the case of male subjects.

In some aspects, the system further comprises an electronic device communicatively linked to the controller, wherein the electronic device is configured to receive a signal based on the sound waves received by the second transducer (or the first transducer in the case where a single transducer is used for stimulation and imaging), from the controller, and to detect or identify one or more anatomical features of the human subject based on the received signal.

In some aspects, the system is configured to detect or identify the one or more anatomical features of the human subject using one or more artificial intelligence (AI) models or machine learning (ML) algorithms.

In some aspects, the controller is configured to a) cause the first transducer to deliver ultrasonic stimulation to a pudendal visceral, a parasympathetic, and/or a sympathetic nerve of a proximal urethra of the human subject, based on the detected or identified one or more anatomical features of the human subject; and/or b) cause an audible, tactile, or visual notification to be generated when the external stimulator comprising the second transducer (or the first transducer in the case where a single transducer is used for stimulation and imaging) is positioned in a location that would enable ultrasonic stimulation of a pudendal visceral, a parasympathetic, and/or a sympathetic nerve of a proximal urethra of the human subject.

In some aspects, the electronic device is communicatively linked to the controller via a wired or wireless connection, and the audible, tactile, or visual notification is provided by an interface of the electronic device.

In some aspects, the one or more anatomical features of the human subject comprise one or more tissues, bones, organs, glands, and/or nerves, of the human subject.

In some aspects, the housing of at least one of the one or more external stimulators is configured to be worn by, placed on, held in proximity to, or affixed to, the human subject while in operation.

In some aspects, the system is configured to operate in a mode that allows for manual activation of at least one of the one or more external stimulators, by the human subject.

In some aspects, the one or more external stimulators are configured to deliver sufficient stimulation to cause the human subject to urinate.

In a second general aspect, the disclosure provides methods of treating any medical conditions or diseases described herein, using the foregoing systems, or any other systems set forth herein. For example, the disclosure provides a method of treating UAB in a subject in need thereof, comprising: stimulating a pudendal visceral, a parasympathetic, and/or a sympathetic nerve of a proximal urethra of a human subject using one or more external stimulators, thereby causing the human subject to urinate, wherein each external stimulator comprises a housing and at least one transducer, and is configured to deliver ultrasonic, electrical, or magnetic stimulation.

In some aspects of the methods described herein, at least one of the one or more external stimulators comprise a transducer configured to deliver ultrasonic stimulation a) at a frequency of about 150-750 kHz, b) with a spatial peak temporal average intensity below 100 W/cm², c) having a variable pulse repetition frequency, and/or d) having a mechanical index below 1.9. In some aspects, stimulation is provided using a duty cycle of about 0-100% (e.g., 10, 20, 30, 40, 50, 60, 70, 80, 90, or 100%, or a percentage within a range bounded by any pair of the foregoing percentages).

In some aspects of the methods described herein, at least one of the one or more external stimulators comprise: a) a first transducer configured to deliver ultrasonic stimulation, and a second transducer configured to transmit and receive sound waves at one or more frequencies within a range of 2 to 15 MHz; or b) a first transducer configured to deliver ultrasonic stimulation and to transmit and receive sound waves at one or more frequencies within a range of 2 to 15 MHz. The controller may be configured to detect or identify one or more anatomical features of the human subject based on sound waves received by the second transducer (or based on sound waves received by the first transducer in the case where a single transducer is used for stimulation and imaging).

In some aspects of the methods described herein, the method further comprises: detecting or identifying one or more anatomical features of the human subject based on sound waves received by the second transducer, prior to stimulating the pudendal visceral, parasympathetic, and/or sympathetic nerve of the proximal urethra of a human subject wherein the detection or identification is performed by a controller or electronic device communicatively linked, directly or indirectly, to the external stimulator comprising the second transducer.

In some aspects of the methods described herein, the controller is configured to a) cause the first transducer to deliver ultrasonic stimulation to the pudendal visceral, parasympathetic, and/or sympathetic nerve of the proximal urethra of the human subject, based on the detected or identified one or more anatomical features of the human subject; and/or b) cause an audible or visual notification to be generated when the external stimulator comprising the second transducer is positioned in a location that would enable ultrasonic stimulation of the pudendal visceral, parasympathetic, and/or sympathetic nerve of a proximal urethra of the human subject.

In some aspects of the methods described herein, the electronic device is communicatively linked to the controller via a wired or wireless connection, and the audible or visual notification is provided by an interface of the electronic device.

In some aspects of the methods described herein, the one or more anatomical features of the human subject comprise one or more tissues, bones, organs, glands, and/or nerves, of the human subject.

In some aspects of the methods described herein, at least one of the one or more external stimulators comprise a transducer at least partially enclosed by a housing, wherein the housing is configured to be worn by, placed on, held in proximity to, or affixed to, the human subject while the external stimulator is in operation.

In some aspects of the methods described herein, the stimulating step is performed in response to input received by a user interface of a controller or electronic device communicatively linked to the one or more external stimulators.

In some aspects of the methods described herein, the stimulating step comprises delivery of sufficient stimulation to cause the human subject to urinate.

In some aspects, the disclosure provides a system for treating UAB, comprising: one or more stimulators, configured to deliver ultrasonic, electrical, and/or magnetic stimulation to at least one nerve of a human subject using a percutaneous lead or catheter; a controller, configured to communicate with the one or more stimulators, and to set or modify one or more parameters of the delivery of the ultrasonic, electrical, or magnetic stimulation by the one or more stimulators; wherein at least one of the one or more stimulators is capable of delivering stimulation to a pudendal visceral, a parasympathetic, and/or a sympathetic nerve of a proximal urethra of the human subject using the percutaneous lead or catheter.

In some aspects, the controller is configured to cause at least one of the one or more stimulators to deliver stimulation to the pudendal visceral, the parasympathetic, and/or the sympathetic nerve of the proximal urethra of the human subject, in response to input provided by the subject. Input may be provided, e.g., using a user interface of a dedicated controller, or a software application executed in an electronic device, capable of communicating with the controller of the system. In some aspects a system may be configured to deliver stimulation in response to input provided by the subject and/or in response to the controller determining one or more parameters of the subject's bladder (e.g., a bladder volume, size, position, or any other metric). Thus, manual and/or automatic stimulation modes are envisioned within the scope of the present disclosure.

In some aspects, the one or more stimulators comprises a plurality of stimulators, said plurality comprising: a) at least a stimulator configured to deliver electrical stimulator using the percutaneous lead or catheter, and b) at least one external stimulator configured to deliver ultrasonic or vibratory stimulation when placed on or in proximity to a skin surface of the subject.

In some aspects, the disclosure provides a method for treating UAB, comprising: stimulating the pudendal visceral, the parasympathetic, and/or the sympathetic nerve of the proximal urethra of the subject using a system comprising at least one stimulator configured to deliver percutaneous stimulation, thereby causing the subject to urinate.

In some aspects, the percutaneous stimulation is provided in response to input provided by the subject. In some aspects, the percutaneous stimulation comprises electrical stimulation delivered to at least one of the nerves described herein, using a percutaneous lead or catheter.

### BRIEF DESCRIPTION OF THE DRAWINGS

FIG. 1 is a block diagram showing an exemplary system according to the disclosure. In this non-limiting example, two external stimulator 101 units are shown, one including a second transducer 103b (e.g., for use in diagnostic imaging), as well as associated system components that may be used in some aspects of the present disclosure.

FIG. 2 is a flowchart illustrating an exemplary method according to the disclosure, including several optional steps.

FIG. 3 is a is a block diagram of various example system components, capable of being used along the lines as described in example implementations in accordance with aspects of the present disclosure.

The exemplary systems and methods shown in the foregoing drawings represent non-limiting examples and do not constrain the scope of the inventions described herein, which are defined solely by the claims.

### DETAILED DESCRIPTION

The detailed description set forth below is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several exemplary aspects according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (“GPUs”), central processing units (“CPUs”), application processors, digital signal processors (“DSPs”), reduced instruction set computing (“RISC”) processors, systems on a chip (“SoC”), baseband processors, field programmable gate arrays (“FPGAs”), programmable logic devices (“PLDs”), application-specific integrated circuits (“ASICs”), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (“RAM”), a read-only memory (“ROM”), an electrically erasable programmable ROM (“EEPROM”), optical disk storage, magnetic disk storage, other magnetic storage devices, combinations of the aforementioned types of computer-readable media, or any other medium that can be used to store computer executable code in the form of instructions or data structures that can be accessed by a computer.

As indicated above, the present disclosure provides systems and methods for treating UAB using one or more external stimulators 101. Prior research has demonstrated that electrical stimulation of the proximal urethra may alleviate symptoms of UAB in animal models. Stimulation at this location involves potentially stimulating, unilaterally and/or bilaterally, the pudendal visceral nerve (Barrington Reflex #2), the parasympathetic nerve (Barrington Reflex #7) and the sympathetic nerve (Barrington Reflex #3), which normally work together to alleviate UAB. Stimulating one of these nerves unilaterally (1 target), or stimulating groups of them unilaterally or bilaterally (up to 6 targets) may also alleviate UAB and produce voiding of the bladder.

UAB patients may be treated by surgical implantation of a stimulator system comprising an IPG and a lead extending from the IPG to one or more of the aforementioned target nerves, where the implanted system is used to stimulate the bladder when voiding is desired. Such systems are described, e.g., in International Patent Application Pub. No. WO 2021/022020A1. While this may be a suitable embodiment for therapy in some cases, a non-invasive or less invasive option is desirable (e.g., to reduce the cost and risk associated with surgery). Accordingly, the present systems and methods utilize one or more external stimulators 101 configured to deliver ultrasonic, electrical, magnetic, and/or vibratory stimulation, or a combination thereof, to a target area (e.g., in proximity to any combination of the nerves described herein) when urination is desired, thereby avoiding the need for surgery. In some aspects, this approach may alternatively be used to verify that a surgical implant may be warranted through a “trial” system based on external stimulation demonstrating effectiveness. In another aspect, this approach may be applied temporarily to a patient who is suffering from UAB acutely (i.e., post-operatively), but not chronically.

In some aspects, focused ultrasonic stimulation is delivered from at least one external stimulator 101. However, other forms of energy may be delivered by other means, e.g., transcutaneously, percutaneously, or via an implantable system. For example, a system according to the disclosure may utilize at least one external stimulator 101 configured to deliver ultrasonic stimulation, and one or more external stimulators 101 (or implanted stimulators) designed to deliver ultrasonic or any other form of stimulation (e.g., electrical stimulation, delivered by an IPG, or vibratory stimulation delivered transcutaneously by a second external stimulator 101). Stimulation of the proximal urethra (involving between 1 and 6 of the target nerves listed herein) may be performed to alleviate UAB and produce bladder voiding on demand. Since voiding is required frequently throughout the day, the stimulation systems described herein may be used by a patient at home, as well as in a hospital setting (e.g., when voiding is challenging and catheterization is performed. For example, a typical use case may be to treat post-operative urinary retention. The present systems may be particularly advantageous where the need is acute (lasting days) and an implant is unwarranted.

It is envisioned that the systems described herein may utilize an external stimulator 101 comprising one or more ultrasonic transducers to allow for diagnostic imaging and delivery of therapeutic stimulation. For example, focused ultrasonic beams may be provided by a first transducer 103a in order to generate a diagnostic medical image or to detect or identify one or more anatomical features of the human subject, while a second transducer 103b (e.g., integrated into the same housing 102 as the first transducer) is used to deliver therapeutic ultrasonic stimulation when the external stimulator 101 is in a suitable position for stimulation of the target nerve(s). In some cases, targeting may not be required to deliver effective stimulation. For example, a system that delivers stimulation at or in proximity to the pubic bone in males and females may not need targeting if the ultrasonic field strength and beam width are set to encompass and stimulate one or more of the target nerves. However, targeting stimulation based on diagnostic imaging, or more generally based on the detection or identification of one or more anatomical features of the human subject, may allow for stimulation to be delivered at lower intensity while still being effective.

If targeting is desired, an ultrasound transducer 103 may be used in a diagnostic imaging mode to identify features in the body that would indicate where the target nerves are likely to be (e.g., if the nerves themselves cannot be imaged). The user (e.g. the patient or a medical practitioner) may then move the transducer 103 around until the ultrasonic beam is in the target area. In some aspects, this process may be automated (e.g., an image analyzer may direct the beam using a servo or some other means, optionally, using one or more artificial intelligence (AI) or machine learning (ML) algorithms or models trained to detect or identify anatomical features. This targeting process may be refined over time by the results (in terms of voiding) each individual voiding session. For example, the external stimulator(s) 101 may include a user interface, or be communicatively linked with a portable or stationary electronic device (106a, 106b) that provides a user interface, allowing the user to provide feedback during or after stimulation (e.g., to identify parameters that result in successful voiding, or that provide an improvement compared to one or more standard or historical parameters, or cause some undesired side effect).

Patients suffering from UAB may or may not be aware of when voiding is necessary (e.g., due to nerve damage or dysfunction). Thus, it is envisioned that in some aspects the systems described herein may utilize a companion application to remind patients when they should consider voiding (e.g., based on a pre-programmed schedule, liquid and/or food consumption information provided by the user or a third party). The companion application may be executed on a portable or stationary electronic device (106a, 106b), such as a patient's mobile phone, tablet, computer, or a dedicated controller. In some aspects, the companion application may take input or biometric data provided by one or more sensors or secondary devices (e.g., obtained using a camera, a voice indication/microphone, etc.). In some aspects, a sensor (e.g., for detecting pressure or distention) may be affixed to the skin in an area over the bladder of the patient and used to provide feedback on when voiding is required. The companion application may be configured to provide a notification (e.g., a visual, tactile, or audio signal, or a combination thereof) when voiding is required. Such notification(s) may be provided by the companion application, or by a communicatively linked device (e.g., a wearable device, a mobile phone, etc.). Any or all of the functionality of the aforementioned companion application may alternatively be performed by a controller integrated into the one or more external stimulators 101 described herein. For example, in some aspects, notifications may be provided by an interface integrated into a housing of the one or more of the external stimulators 101). Similarly, any of the sensor data or functionality described herein may be performed by one or more sensors attached to, integrated into, or located partially or wholly within, a housing of the one or more external stimulators described herein. For example, an external stimulator 101 may be configured to be worn by a user, or to be held in proximity to a skin surface of the user, allowing for imaging of the user's bladder to be performed using at least one transducer of the external stimulator. Such configurations may be used to provide feedback to the user and/or to collect data regarding bladder volume, to monitor the effectiveness of UAB therapies, etc.

While the present disclosure is primarily directed to systems that utilize external stimulators 101, it is envisioned that in some aspects a percutaneous device may be used instead of or in addition to an external stimulator 101. For example, post-operative patients suffering from urinary retention, or other patients that may have a need for UAB stimulation for a fixed period of time (e.g., not chronically), may benefit from a percutaneous device configured to deliver energy sufficient to stimulate any of the nerves described herein when voiding is necessary. In some embodiments, a percutaneous lead or catheter may be positioned in contact with or in proximity to the proximal urethra and the electrode(s) may be placed using a non-surgical approach. The lead or catheter may simply be withdrawn when there is no longer a need for assisted voiding. A percutaneous lead of this sort may be used to deliver electrical stimulation alone, or in combination with stimulation via one or more external stimulators 101 (e.g., in combination with ultrasonic stimulation provided by at least one external stimulator 101).

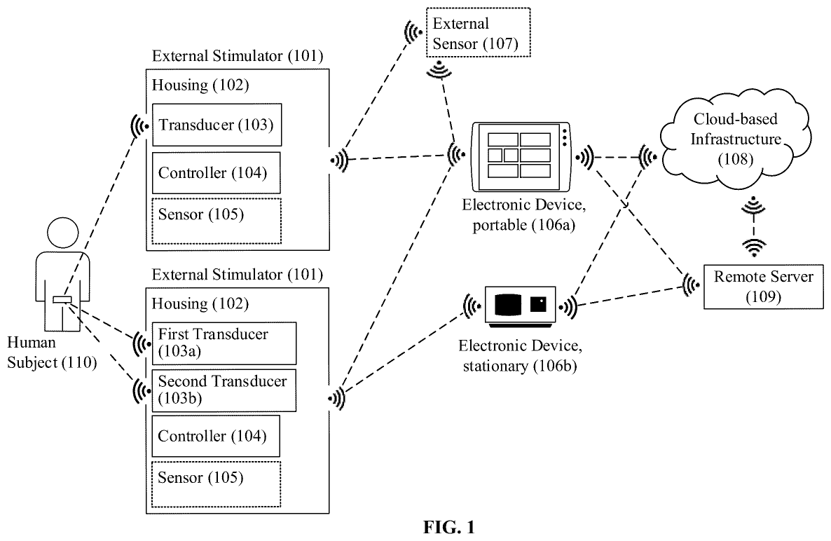

FIG. 1 is a block diagram of an exemplary system according to the present disclosure. This particular example illustrates a stimulation system comprising two external stimulators 101, each comprising a housing 102, at least one transducer 103, 103a, 103b, a controller 104, and optionally a sensor 105. The second external stimulator 101 (bottom) demonstrates the use of two transducers 103a, 103b, allowing for diagnostic imaging or the detection and/or identification of one or more anatomical features of the human subject 110, using the first transducer 103a, while therapeutic stimulation is delivered by the second transducer 103b. As explained above, such configurations may advantageously allow for targeted delivery of stimulation, when needed (e.g., to stimulate one or more of the nerves described herein). In this example, two transducers are used to achieve this functionality. However, it is envisioned that in other embodiments a single transducer may be sufficient (e.g., a single transducer may allow for transmission and receipt of ultrasonic stimulation at multiple frequencies simultaneously, or by rapid cycling between a diagnostic and therapeutic frequency). In either case, as shown by this figure the one or more external stimulators 101 may be communicatively linked to one or more electronic devices, such as a portable 106a or stationary 106b electronic device. Electronic devices may include, e.g., a smart phone, tablet computer, desktop or laptop computer, or a dedicated system controller. The electronic device will typically include a user interface, e.g., a graphical user interface, which may be used by the patient or a third party (e.g., a medical practitioner) to view, modify, or adjust one or more parameters of the stimulation provided by the systems described herein, and/or to provide feedback regarding stimulation, as explained above.

The exemplary system shown in FIG. 1 also illustrates the use of sensors, including sensors 105 integrated into the housing of the external stimulators 101, and an external sensor 107. In this case, these optional sensors are all external. However, internal sensors may also be used. Sensor data may be collected to measure one or more biometric parameters of the subject, e.g., in order to trigger, optimize, or evaluate the effects of stimulation provided by the one or more external stimulators 101. For example, an external sensor 107 placed on a skin surface of the subject's lower abdomen may be used to measure pressure or distention caused by bladder volume, and sent to a controller 104 of an external stimulator 101 or to an associated electronic device 106a, 106b. Sensor data may be used to augment the present systems by providing another signal that may be taken into account when determining when to apply stimulation (e.g., a controller 104 may be configured to trigger stimulation based on sensor data indicating that bladder volume has likely exceeded a predetermined threshold).

Stimulation may be activated by the controller 104 integrated into an external stimulator 101 or by an associated electronic device 106a, 106b. In some cases, the systems described herein may allow for sensor or other data to be sent to a remote server 109 (e.g., directly from an electronic device 106a, 106b) or via a cloud-based infrastructure 108 or other infrastructure), for logging or further processing. For example, sensor data obtained from one or more sensors 105, 107, or diagnostic medical imaging data collected from a transducer 103, 103a, 103b of an external stimulator 101, may be sent to a remote server for processing in order to evaluate treatment effectiveness or to optimize treatment parameters. In some aspects, AI or ML models/algorithms may be used to select or optimize treatment parameters (e.g., intensity, duty cycle, frequency, or positioning of an external stimulator).

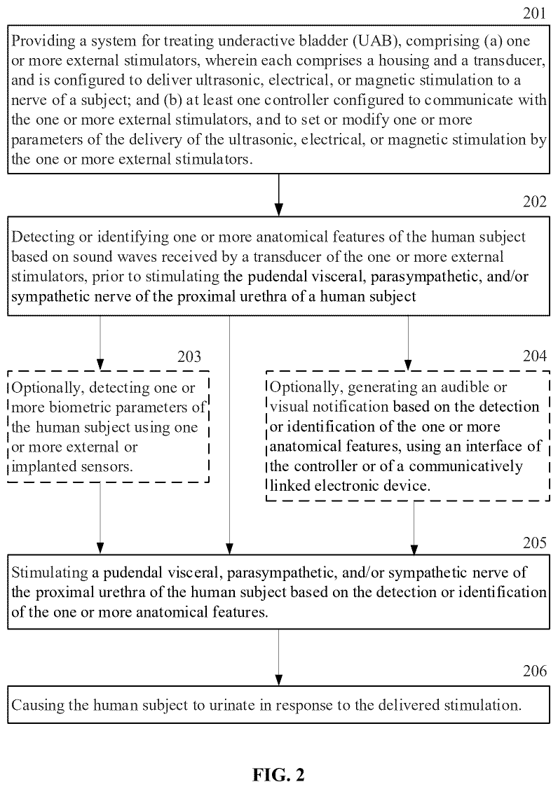

FIG. 2 is a flowchart illustrating an exemplary method for treating UAB according to the disclosure. In this case, the method includes providing a system comprising (a) one or more external stimulators, wherein each comprises a housing and a transducer, and is configured to deliver ultrasonic, electrical, or magnetic stimulation to a nerve of a subject; and (b) at least one controller configured to communicate with the one or more external stimulators, and to set or modify one or more parameters of the delivery of the ultrasonic, electrical, or magnetic stimulation by the one or more external stimulators (201), Next, one or more anatomical features of the human subject are detected or identified based on sound waves received by a transducer of the one or more external stimulators, prior to stimulating the pudendal visceral, parasympathetic, and/or sympathetic nerve of the proximal urethra of a human subject (202).

At this stage, the exemplary method diverges along three paths. Optionally, one or more biometric parameters of the human subject may be detected using one or more external or implanted sensors (203). For example, pressure or distention caused by bladder volume may be detected using an external sensor 107 placed on a skin surface of the subject's abdomen. In other aspects, additional or alternative sensors may be used. Sensor data may be used, e.g., to determine one or more treatment parameters (e.g., intensity, duty cycle, and/or location of stimulation). As a second optional step, an audible, visual, or other notification (e.g., a tactile notification, or any combination thereof) may be generated based on the detection or identification of the one or more anatomical features, using an interface of the controller 104 of an external stimulator 101 or of a communicatively linked electronic device 106a, 106b (204). This notification may be used to alert a user that an external stimulator 101 of the systems described herein is placed in a position suitable for delivering stimulation to a target area (or any one or combination of the nerves described herein).

Stimulation of a pudendal visceral, parasympathetic, and/or sympathetic nerve of the proximal urethra of the human subject based on the detection or identification of the one or more anatomical features may then proceed (205), directly or following either of the optional steps shown in this figure, thereby causing the human subject to urinate in response to the delivered stimulation (206).

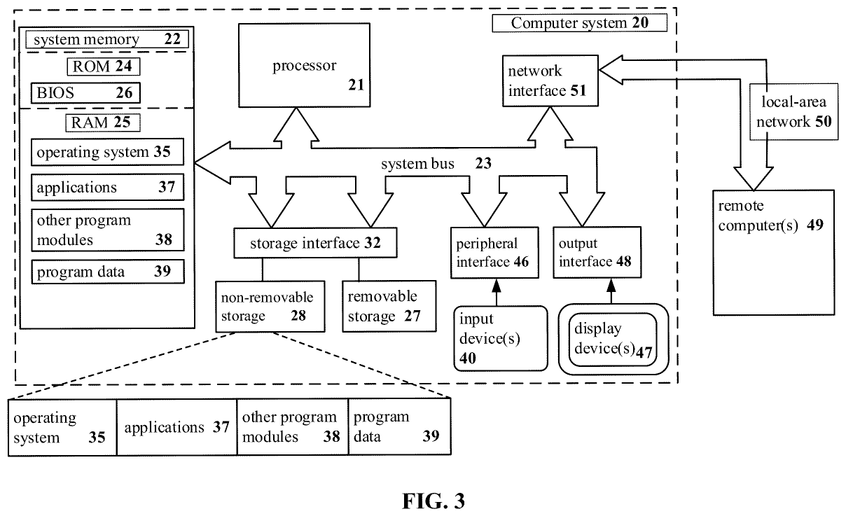

Aspects of the present disclosure may be implemented using hardware, software, or a combination thereof and may be implemented in one or more computer systems or other processing systems. In an aspect of the present disclosure, features are directed toward one or more computer systems capable of carrying out the functionality described herein. FIG. 3 is a block diagram illustrating an example of a computer system 20 which may be used to implement aspects of the systems and methods described herein. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I2C, and other suitable interconnects. The central processing unit 21 (also referred to as a processor) can include a single or multiple sets of processors having single or multiple cores. The processor 21 may execute one or more computer-executable codes implementing the techniques of the present disclosure. For example, any of commands/steps discussed in this specification, or shown in the accompanying drawings, may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object-oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain aspects of the present disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the present disclosure to be practiced otherwise than specifically described herein. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing aspects of the present disclosure (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or “exemplary” language (e.g., “such as”) provided herein is intended merely to better illuminate the present disclosure and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

### EMBODIMENTS

Embodiment 1. A system for treating underactive bladder (UAB), comprising: one or more external stimulators, wherein each comprises a housing and a transducer, and is configured to deliver ultrasonic, electrical, and/or magnetic stimulation to at least one nerve of a human subject; a controller, configured to communicate with the one or more external stimulators, and to set or modify one or more parameters of the delivery of the ultrasonic, electrical, or magnetic stimulation by the one or more external stimulators; wherein at least one of the one or more external stimulators is capable of delivering stimulation to a pudendal visceral, a parasympathetic, and/or a sympathetic nerve of a proximal urethra of the human subject when placed on or in proximity to a skin surface of the human subject.

Embodiment 2. The system of Embodiment 1, wherein the one or more external stimulators comprise at least one transducer configured to deliver ultrasonic stimulation.

Embodiment 3. The system of Embodiment 2, wherein the at least one transducer configured to deliver ultrasonic stimulation is configured to deliver stimulation: a) at a frequency of 150-750 kHz, b) with a spatial peak temporal average intensity below 100 W/cm2 , c) having a variable pulse repetition frequency, d) having a mechanical index below 1.9, and/or e) using a duty cycle of 0-100%.

Embodiment 4. The system of any one of Embodiments 1-3, wherein at least one of the one or more external stimulators comprise: a) a first transducer configured to deliver ultrasonic stimulation, and a second transducer configured to transmit and receive sound waves at one or more frequencies within a range of 2 to 15 MHz, wherein the controller is configured to detect or identify one or more anatomical features of the human subject based on sound waves received by the second transducer; or b) a first transducer configured to deliver ultrasonic stimulation and to transmit and receive sound waves at one or more frequencies within a range of 2 to 15 MHz, wherein the controller is configured to detect or identify one or more anatomical features of the human subject based on sound waves received by the first transducer.

Embodiment 5. The system of Embodiment 4, wherein the controller is configured to detect or identify one or more anatomical features of the human subject based on sound waves received by the second transducer.

Embodiment 6. The system of Embodiment 4, further comprising an electronic device communicatively linked to the controller, wherein the electronic device is configured to receive a signal based on the sound waves received by the second transducer, from the controller, and to detect or identify one or more anatomical features of the human subject based on the received signal.

Embodiment 7. The system of Embodiments 5 or 6, wherein the system is configured to detect or identify the one or more anatomical features of the human subject using one or more artificial intelligence (AI) models or machine learning (ML) algorithms.

Embodiment 8. The system of any one of Embodiments 5-7, wherein the controller is configured to a) cause the first transducer to deliver ultrasonic stimulation to a pudendal visceral, a parasympathetic, and/or a sympathetic nerve of a proximal urethra of the human subject, based on the detected or identified one or more anatomical features of the human subject; and/or b) cause an audible, tactile, or visual notification to be generated when the external stimulator comprising the second transducer is positioned in a location that would enable ultrasonic stimulation of a pudendal visceral, a parasympathetic, and/or a sympathetic nerve of a proximal urethra of the human subject.

Embodiment 9. The system of Embodiment 8, wherein: a) the audible, tactile, or visual notification is provided by an interface of the one or more external stimulators; or b) the electronic device is communicatively linked to the controller via a wired or wireless connection, and the audible, tactile, or visual notification is provided by an interface of the electronic device.

Embodiment 10. The system of any one of Embodiments 5-9, wherein the one or more anatomical features of the human subject comprise one or more tissues, bones, organs, glands and/or nerves, of the human subject.

Embodiment 11. The system of any one of Embodiments 1-10 wherein at least one of the one or more external stimulators is configured to be worn by, placed on, held in proximity to, or affixed to, the human subject while in operation.

Embodiment 12. The system of any one of Embodiments 1-11, wherein the system is configured to operate in a mode that allows for manual activation of at least one of the one or more external stimulators, by the human subject.

Embodiment 13. The system of any one of Embodiments 1-12, wherein the one or more external stimulators are configured to deliver sufficient stimulation to cause the human subject to urinate.

Embodiment 14. A method for treating UAB, comprising: stimulating a pudendal visceral, a parasympathetic, and/or a sympathetic nerve of a proximal urethra of a human subject using one or more external stimulators, thereby causing the human subject to urinate, wherein each external stimulator comprises a housing and at least one transducer, and is configured to deliver ultrasonic, electrical, and/or magnetic stimulation.

Embodiment 15. The method of Embodiment 14, wherein at least one of the one or more external stimulators comprise a transducer configured to deliver ultrasonic stimulation: a) at a frequency of 150-750 kHz, b) with a spatial peak temporal average intensity below 100 W/cm2, c) having a variable pulse repetition frequency, d) having a mechanical index below 1.9, and/or e) using a duty cycle of 0-100%.

Embodiment 16. The method of Embodiments 14 or 15, wherein at least one of the one or more external stimulators comprise: a) a first transducer configured to deliver ultrasonic stimulation, and a second transducer configured to transmit and receive sound waves at one or more frequencies within a range of 2 to 15 MHz, wherein the controller is configured to detect or identify one or more anatomical features of the human subject based on sound waves received by the second transducer; or b) a first transducer configured to deliver ultrasonic stimulation and to transmit and receive sound waves at one or more frequencies within a range of 2 to 15 MHz, wherein the controller is configured to detect or identify one or more anatomical features of the human subject based on sound waves received by the first transducer.

Embodiment 17. The method of Embodiment 16, further comprising: detecting or identifying one or more anatomical features of the human subject based on sound waves received by the second transducer, prior to stimulating the pudendal visceral, parasympathetic, and/or sympathetic nerve of the proximal urethra of a human subject; wherein the detection or identification is performed by a controller or electronic device communicatively linked, directly or indirectly, to the external stimulator comprising the second transducer.

Embodiment 18. The method of Embodiment 17, wherein the controller is configured to a) cause the first transducer to deliver ultrasonic stimulation to the pudendal visceral, parasympathetic, and/or sympathetic nerve of the proximal urethra of the human subject, based on the detected or identified one or more anatomical features of the human subject; and/or b) cause an audible or visual notification to be generated when the external stimulator comprising the second transducer is positioned in a location that would enable ultrasonic stimulation of the pudendal visceral, parasympathetic, and/or sympathetic nerve of a proximal urethra of the human subject.

Embodiment 19. The method of Embodiment 18, wherein: a) the audible, tactile, or visual notification is provided by an interface of the one or more external stimulators; or b) the electronic device is communicatively linked to the controller via a wired or wireless connection, and the audible or visual notification is provided by an interface of the electronic device.

Embodiment 20. The method of any one of Embodiments 17-19, wherein the one or more anatomical features of the human subject comprise one or more tissues, bones, organs, and/or nerves, of the human subject.

Embodiment 21. The method of any one of Embodiments 14-20, wherein at least one of the one or more external stimulators comprise a transducer at least partially enclosed by a housing, wherein the housing is configured to be worn by, placed on, or affixed to, the human subject while the external stimulator is in operation.

Embodiment 22. The method of any one of Embodiments 14-21, wherein the stimulating step is performed in response to input received by a user interface of a controller or electronic device communicatively linked to the one or more external stimulators.

Embodiment 23. The method of any one of Embodiments 14-21, wherein the stimulating step comprises delivery of sufficient stimulation to cause the human subject to urinate.

Embodiment 24. A system for treating UAB, comprising: one or more stimulators, configured to deliver ultrasonic, electrical, and/or magnetic stimulation to at least one nerve of a human subject using a percutaneous lead or catheter; a controller, configured to communicate with the one or more stimulators, and to set or modify one or more parameters of the delivery of the ultrasonic, electrical, and/or magnetic stimulation by the one or more stimulators; wherein at least one of the one or more stimulators is capable of delivering stimulation to a pudendal visceral, a parasympathetic, and/or a sympathetic nerve of a proximal urethra of the human subject using the percutaneous lead or catheter.

Embodiment 25. The system of Embodiment 24, wherein the controller is configured to cause at least one of the one or more stimulators to deliver stimulation to the pudendal visceral, the parasympathetic, and/or the sympathetic nerve of the proximal urethra of the human subject, in response to input provided by the subject.

Embodiment 26. The system of Embodiment 24, wherein the one or more stimulators comprises a plurality of stimulators, said plurality comprising: at least one stimulator configured to deliver electrical stimulator using the percutaneous lead or catheter, and at least one external stimulator configured to deliver ultrasonic or vibratory stimulation when placed on or in proximity to a skin surface of the subject.

Embodiment 27. A method for treating UAB, comprising: stimulating the pudendal visceral, the parasympathetic, and/or the sympathetic nerve of the proximal urethra of the subject using the system of Embodiment 24, in response to a) input provided by the subject and/or b) a determination as to a volume of the subject's bladder, thereby causing the human subject to urinate.

1. A system for treating underactive bladder (UAB), comprising:
one or more external stimulators, wherein each comprises a housing and a transducer, and is configured to deliver ultrasonic, electrical, and/or magnetic stimulation to at least one nerve of a human subject;
a controller, configured to communicate with the one or more external stimulators, and to set or modify one or more parameters of the delivery of the ultrasonic, electrical, and/or magnetic stimulation by the one or more external stimulators;
wherein at least one of the one or more external stimulators is capable of delivering stimulation to a pudendal visceral, a parasympathetic, and/or a sympathetic nerve of a proximal urethra of the human subject when placed on or in proximity to a skin surface of the human subject.

2. The system of claim 1, wherein the one or more external stimulators comprises:
a) at least one transducer configured to deliver ultrasonic stimulation;
b) a first transducer configured to deliver ultrasonic stimulation, and a second transducer configured to transmit and receive sound waves at one or more frequencies within a range of 2 to 15 MHz, wherein the controller is configured to detect or identify one or more anatomical features of the human subject based on sound waves received by the second transducer; or
b) a first transducer configured to deliver ultrasonic stimulation and to transmit and receive sound waves at one or more frequencies within a range of 2 to 15 MHz, wherein the controller is configured to detect or identify one or more anatomical features of the human subject based on sound waves received by the first transducer.

3. The system of claim 2, wherein the at least one transducer configured to deliver ultrasonic stimulation is configured to deliver stimulation: a) at a frequency of 150-750 kHz, b) with a spatial peak temporal average intensity below 100 W/cm², c) having a variable pulse repetition frequency, d) having a mechanical index below 1.9, and/or e) using a duty cycle of 0-100%.

4. The system of claim 2, wherein the controller is configured to detect or identify one or more anatomical features of the human subject based on sound waves received by the second transducer.

5. The system of claim 2, further comprising an electronic device communicatively linked to the controller, wherein the electronic device is configured to receive a signal based on the sound waves received by the second transducer, from the controller, and to detect or identify one or more anatomical features of the human subject based on the received signal.

6. The system of claim 2, wherein the controller is configured to
a) cause the first transducer to deliver ultrasonic stimulation to a pudendal visceral, a parasympathetic, and/or a sympathetic nerve of a proximal urethra of the human subject, based on the detected or identified one or more anatomical features of the human subject; and/or
b) cause an audible, tactile, and/or visual notification to be generated when the external stimulator comprising the first transducer is positioned in a location that would enable ultrasonic stimulation of a pudendal visceral, a parasympathetic, and/or a sympathetic nerve of a proximal urethra of the human subject.

7. The system of claim 6, wherein:
a) the audible, tactile, and/or visual notification is provided by an interface of the one or more external stimulators; or
b) the electronic device is communicatively linked to the controller via a wired or wireless connection, and the audible, tactile, or visual notification is provided by an interface of the electronic device.

8. The system of claim 4, wherein the one or more anatomical features of the human subject comprise one or more tissues, bones, organs, glands and/or nerves, of the human subject.

9. The system of claim 2, wherein
a) at least one of the one or more external stimulators is configured to be worn by, placed on, held in proximity to, or affixed to, the human subject while in operation;
b) the system is configured to operate in a mode that allows for manual activation of at least one of the one or more external stimulators, by the human subject; and/or
c) the one or more external stimulators are configured to deliver sufficient stimulation to cause the human subject to urinate.

10. A method for treating underactive bladder (UAB), comprising:
stimulating a pudendal visceral, a parasympathetic, and/or a sympathetic nerve of a proximal urethra of a human subject using one or more external stimulators, thereby causing the human subject to urinate,
wherein each external stimulator comprises a housing and at least one transducer, and is configured to deliver ultrasonic, electrical, and/or magnetic stimulation.

11. The method of claim 10, wherein at least one of the one or more external stimulators comprises a transducer configured to deliver ultrasonic stimulation: a) at a frequency of 150-750 kHz, b) with a spatial peak temporal average intensity below 100 W/cm², c) having a variable pulse repetition frequency, d) having a mechanical index below 1.9, and/or e) using a duty cycle of 0-100%.

12. The method of claim 10, wherein at least one of the one or more external stimulators comprises:
a) a first transducer configured to deliver ultrasonic stimulation, and a second transducer configured to transmit and receive sound waves at one or more frequencies within a range of 2 to 15 MHz, wherein the controller is configured to detect or identify one or more anatomical features of the human subject based on sound waves received by the second transducer; or
b) a first transducer configured to deliver ultrasonic stimulation and to transmit and receive sound waves at one or more frequencies within a range of 2 to 15 MHz, wherein the controller is configured to detect or identify one or more anatomical features of the human subject based on sound waves received by the first transducer.

13. The method of claim 12, further comprising a step of:
detecting or identifying one or more anatomical features of the human subject based on sound waves received by the second transducer, prior to stimulating the pudendal visceral, parasympathetic, and/or sympathetic nerve of the proximal urethra of a human subject, said one or more anatomical features of the human subject comprising one or more tissues, bones, organs, and/or nerves, of the human subject;
wherein the detection or identification is performed by a controller or electronic device communicatively linked, directly or indirectly, to the external stimulator comprising the second transducer.

14. The method of claim 13, wherein the controller is configured to
a) cause the first transducer to deliver ultrasonic stimulation to the pudendal visceral, parasympathetic, and/or sympathetic nerve of the proximal urethra of the human subject, based on the detected or identified one or more anatomical features of the human subject; and/or
b) cause an audible or visual notification to be generated when the external stimulator comprising the first transducer is positioned in a location that would enable ultrasonic stimulation of the pudendal visceral, parasympathetic, and/or sympathetic nerve of a proximal urethra of the human subject.

15. The method of claim 14, wherein
a) the audible, tactile, and/or visual notification is provided by an interface of the one or more external stimulators; or
b) the electronic device is communicatively linked to the controller via a wired or wireless connection, and the audible or visual notification is provided by an interface of the electronic device.

16. The method of claim 10, wherein:
a) at least one of the one or more external stimulators comprises a transducer at least partially enclosed by a housing, wherein the housing is configured to be worn by, placed on, or affixed to, the human subject while the external stimulator is in operation;
b) the stimulating step is performed in response to input received by a user interface of a controller or electronic device communicatively linked to the one or more external stimulators; and/or
c) the stimulating step comprises delivery of sufficient stimulation to cause the human subject to urinate.

17. A system for treating underactive bladder (UAB), comprising:
one or more stimulators, configured to deliver ultrasonic, electrical, and/or magnetic stimulation to at least one nerve of a human subject using a percutaneous lead or catheter;
a controller, configured to communicate with the one or more stimulators, and to set or modify one or more parameters of the delivery of the ultrasonic, electrical, and/or magnetic stimulation by the one or more stimulators;
wherein at least one of the one or more stimulators is capable of delivering stimulation to a pudendal visceral, a parasympathetic, and/or a sympathetic nerve of a proximal urethra of the human subject using the percutaneous lead or catheter.

18. The system of claim 17, wherein the controller is configured to cause at least one of the one or more stimulators to deliver stimulation to the pudendal visceral, the parasympathetic, and/or the sympathetic nerve of the proximal urethra of the human subject, in response to a) input provided by the subject and/or a determination as to a volume of the subject's bladder.

19. The system of claim 17, wherein the one or more stimulators comprises a plurality of stimulators, said plurality comprising:
at least one stimulator configured to deliver electrical stimulator using the percutaneous lead or catheter, and
at least one external stimulator configured to deliver ultrasonic and/or vibratory stimulation when placed on or in proximity to a skin surface of the subject.

20. A method for treating underactive bladder (UAB), comprising:
stimulating the pudendal visceral, the parasympathetic, and/or the sympathetic nerve of the proximal urethra of the subject using the system of claim 17, in response to a) input provided by the subject and/or b) a determination as to a volume of the subject's bladder, thereby causing the human subject to urinate.
